(19) United States
(12) Patent Application Publication
(10) Pub. No.: US 2010/0094238 A1
(43) Pub. Date: Apr. 15, 2010

(54) Biodegradable tampon disposal bag

(76) Inventor: Anthony J. Scarano, Brooklyn, NY (US)

Correspondence Address: LITMAN LAW OFFICES, LTD., POST OFFICE BOX 41200, SOUTH STATION, ARLINGTON, VA, 22204, US

(21) Appl. No.: 12/588,162

(22) Filed: Oct. 6, 2009

Related U.S. Application Data
(60) Provisional application No. 61/136,899, filed on Oct. 10, 2008.

(51) Int. Cl. A61F 13/551; B65D 30/02

(52) U.S. Cl. 604/385.13; 383/1

(57) ABSTRACT

The biodegradable tampon disposal bag is a biodegradable, fluid resistant and sealable package for receiving used tampons and other feminine hygiene products for disposal thereof. The tampon disposal bag is formed as an envelope having a pair of longitudinally opposed, closed side edges, an open upper end, and a closed lower end. A flap is secured to the open upper end of the envelope for covering and sealing the open upper end when a used tampon is received within the open interior of the envelope. An adhesive seal or the like is provided on the flap, for securing the flap against the envelope once the tampon has been received therein. The envelope and the flap are both formed from a biodegradable and fluid resistant material, such as low-density polyethylene.

### CROSS-REFERENCE TO RELATED APPLICATION

This application claims the benefit of U.S. Provisional Patent Application Ser. No. 61/136,899, filed Oct. 10, 2008.

### BACKGROUND OF THE INVENTION

1. Field of the Invention

The present invention relates to packages for receiving used hygiene products for disposal thereof, and particularly to a biodegradable tampon disposal bag that provides a fluid resistant envelope dimensioned and configured for receiving a used tampon or other feminine hygiene product.

2. Description of the Related Art

Women are often faced with the challenge of disposing of used feminine hygiene products, such as tampons, sanitary napkins and the like. A typical method of disposal in public restrooms and the like involves wrapping the used sanitary napkin or tampon in an awkward bundle of toilet paper. This, however, does not provide a secure wrapping of the article, thus creating an unsanitary and messy package.

Often, public restrooms in restaurants, stores, and in places of work do not have convenient waste receptacles in the toilet stalls. Even when such receptacles are provided, many women choose not to use them, due to health and safety reasons. One unpleasant option is to remove the article from the restroom and carry the article until it can be disposed of. Alternatively, a bag may be provided and used for disposal of the hygiene product.

A typical bag used for this purpose would be formed from plastic, so as to create a fluid resistant container. However, plastics represent a large environmental concern, as most plastics are non-biodegradable, and the purpose of this particular bag is disposal. Further, prior packages for disposal are typically flushed in toilets, which often results in plumbing backups, blockages and other problems. It would be desirable to provide a package that alleviates such problems. Thus, a tampon disposal bag solving the aforementioned problems is desired.

### SUMMARY OF THE INVENTION

The biodegradable tampon disposal bag is a biodegradable, fluid resistant and sealable package for receiving used tampons and other feminine hygiene products for disposal thereof. The tampon disposal bag includes an envelope having a pair of longitudinally opposed, closed side edges, an open upper end, and a closed lower end. A flap is secured to the open upper end of the envelope for covering and sealing the open upper end when a used tampon is received within the open interior of the envelope.

An adhesive seal or the like is provided on the flap, for securing the flap against the envelope once the tampon has been received therein. The envelope and the flap are both formed from a biodegradable and fluid resistant material, such as low-density polyethylene.

These and other features of the present invention will become readily apparent upon further review of the following specification and drawings.

### BRIEF DESCRIPTION OF THE DRAWINGS

FIG. 1 is a perspective view of a tampon disposal bag according to the present invention.

FIG. 2 is a front plan view of the tampon disposal bag according to the present invention.

FIG. 3 is a rear plan view of the tampon disposal bag according to the present invention.

FIG. 4 is a perspective attachment view of the tampon disposal bag according to the present invention.

FIG. 5 is a perspective view of the tampon disposal bag in a sealed, closed configuration.

Similar reference characters denote corresponding features consistently throughout the attached drawings.

### DETAILED DESCRIPTION OF THE PREFERRED EMBODIMENTS

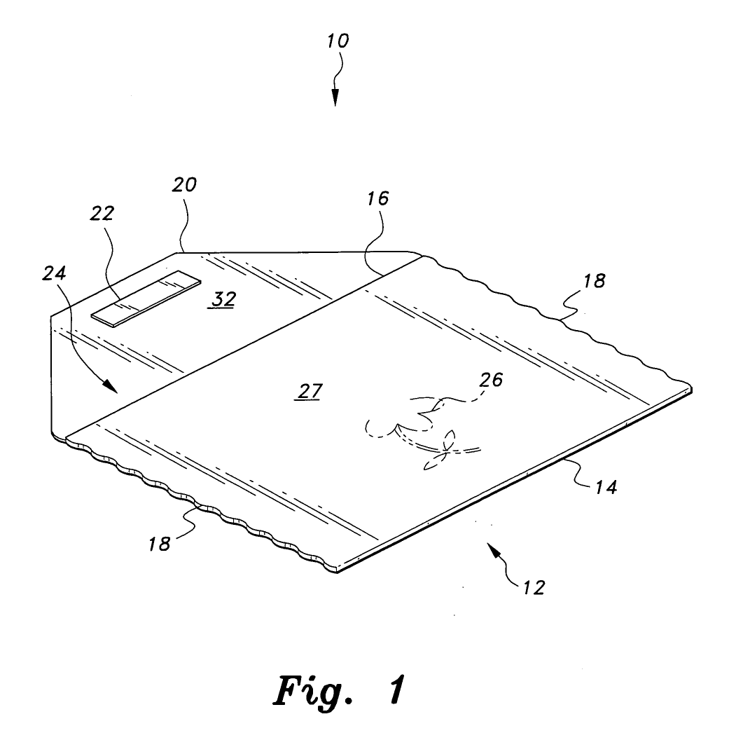

The tampon disposal bag 10 is a biodegradable, fluid resistant and sealable package for receiving used tampons and other feminine hygiene products for disposal thereof. As best shown in FIGS. 1-3, the tampon disposal bag 10 is formed as an envelope 12 having a pair of longitudinally opposed, closed side edges 18, an open upper end 16, and a closed lower end 14. The envelope 12 includes a front face 27 and a rear face 28, both of which are substantially rectangular in the preferred embodiment. Although shown as being substantially rectangular, it should be understood that envelope 12 may have any desired shape. Similarly, the side edges 18 are shown as being sinusoidal, scalloped or undulating, though the shape of the side edges 18 may vary. The envelope 12 may have any desired size, with exemplary dimensions including a longitudinal length of approximately 5½ inches, and a lateral height of approximately 4½ inches.

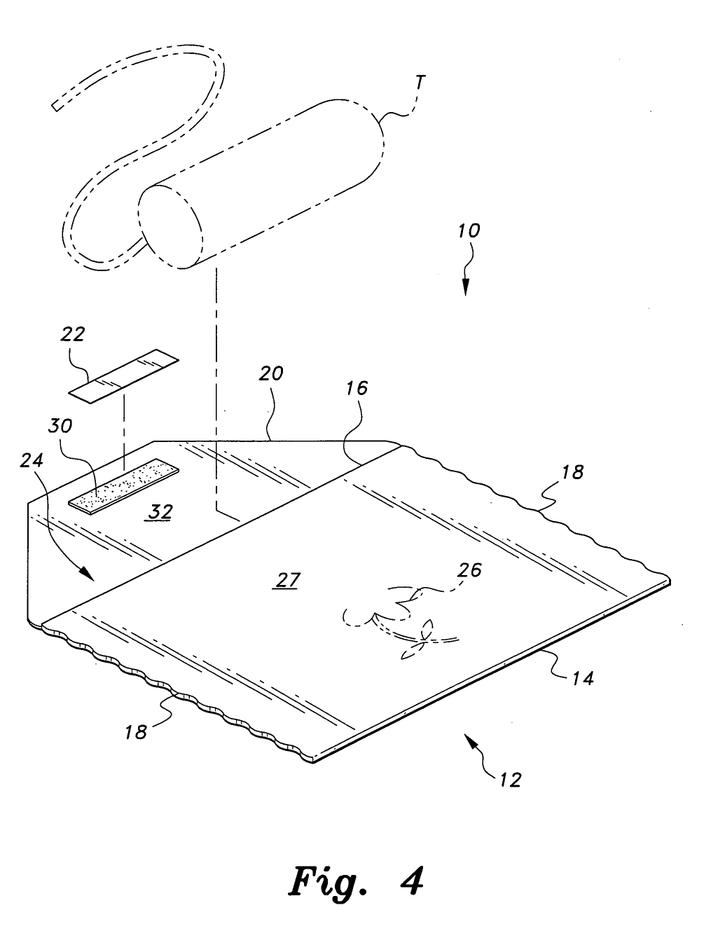

A flap 20 is secured to the open upper end 16 of the envelope 12 for covering and sealing the open upper end 16 when a used tampon T is received within the open interior 24 of the envelope 12 (shown in FIG. 4). Any suitable closure may be used for sealing the front face 32 of flap 20 against the front face 27 of envelope 12 when a tampon T is received within the open interior region 24. In the preferred embodiment, pressure-sensitive adhesive 30 is applied to front face 32 of flap 20, and is covered with a release strip 22 to be removed at the time of use.

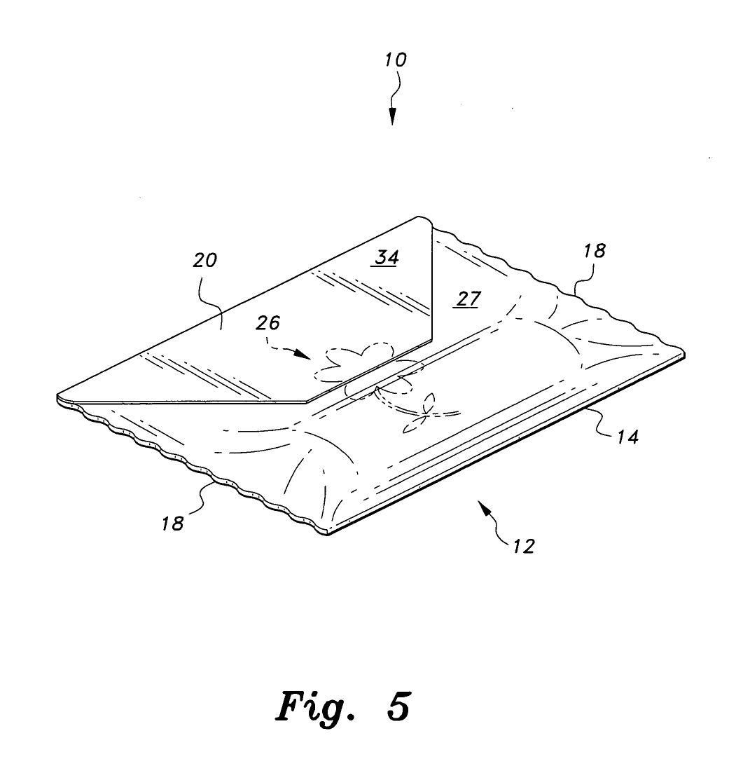

Using the exemplary dimensions given above for the envelope 12, the flap may extend downwardly, when in the sealed position of FIG. 5, approximately two inches. Using these exemplary dimensions, the adhesive layer 30 preferably extends longitudinally approximately one inch, centered with respect to the flap 20. As shown, the flap 20 is preferably substantially trapezoidal, and the adhesive layer 30 is preferably formed adjacent the smaller, upper edge of flap 20. With the given exemplary dimensions for the envelope 12 and the flap 20, the open interior region 24 is capable of storing one or two used, conventional tampons and a tampon applicator, or a used sanitary napkin.

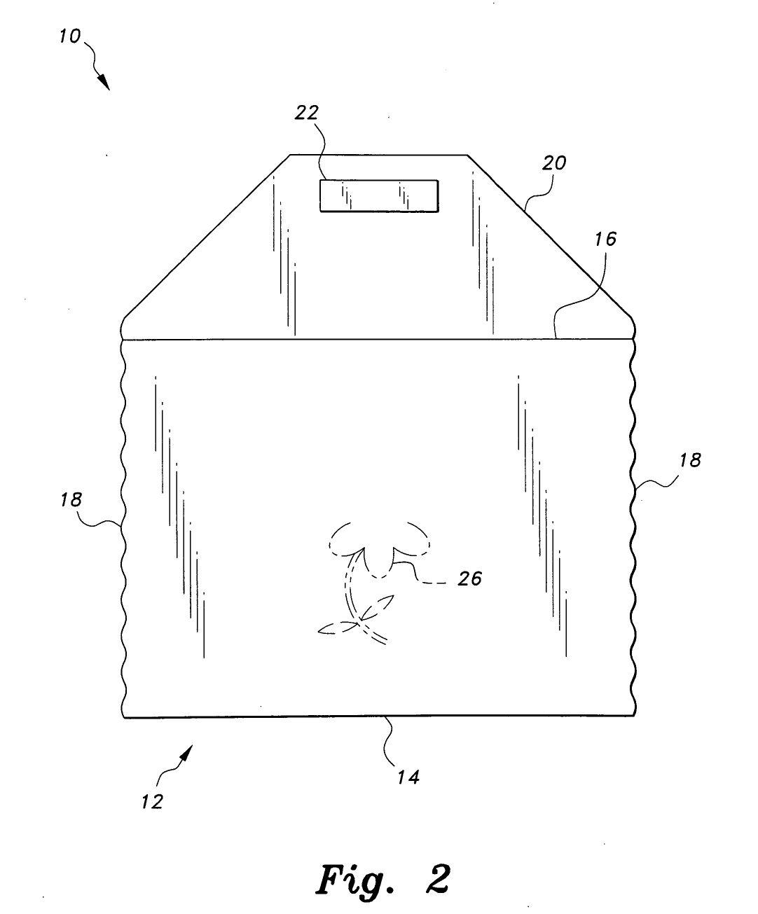

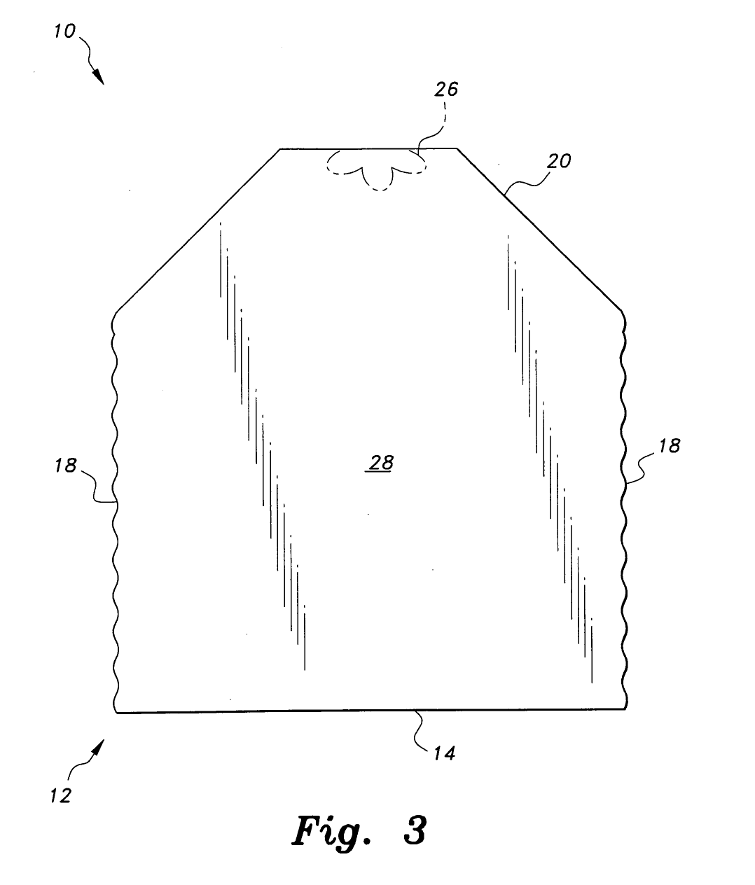

The envelope 12 and the flap 20 are both formed from a biodegradable, tear-resistant and fluid resistant material, such as low-density polyethylene. Additionally, as shown, indicia 26 may be formed on bag 10. As shown in FIG. 2, the exemplary floral design may be partially formed on the front face 27 of the envelope 12, and, as shown in FIG. 3, the completion of the design may be formed on the rear face 34 of the flap 20. Thus, when the bag 10 is sealed, as in FIG. 5, the completed floral design indicia 26 is displayed. This guides the user to properly align the flap 20 for a complete seal of the open end 16.

The flap 20 and the envelope 12 are preferably opaque, so as not to reveal the interior contents, and may be of any desired color. For example, navy blue or burgundy color biodegradable plastic may be used in their formation. Prior to use, as in FIG. 1, the bag 10 preferably has a thickness of approximately two millimeters, thus allowing multiple bags 10 to be stored in an easily transportable pack.

It is to be understood that the present invention is not limited to the embodiment described above, but encompasses any and all embodiments within the scope of the following claims.

I claim:

1. A biodegradable hygiene product disposal bag, comprising:
an envelope having a pair of longitudinally opposed, closed side edges, an open upper end, and a closed lower end, the envelope defining an open interior region adapted for receiving a used hygiene product;
a flap secured to the open upper end of the envelope, the envelope and the flap being formed from a biodegradable and fluid resistant material; and
means for covering and sealing the open upper end of the envelope with the flap.

2. The biodegradable hygiene product disposal bag as recited in claim 1, wherein said envelope and said flap are formed from low-density polyethylene.

3. The biodegradable hygiene product disposal bag as recited in claim 1, wherein said envelope is substantially rectangular.

4. The biodegradable hygiene product disposal bag as recited in claim 3, wherein each said closed side edge is substantially sinusoidal in shape.

5. The biodegradable hygiene product disposal bag as recited in claim 3, wherein said flap is substantially trapezoidal.

6. The biodegradable hygiene product disposal bag as recited in claim 5, wherein first and second sets of indicia are formed on an outer surface of said flap and on a rear surface of said envelope, respectively.

7. The biodegradable hygiene product disposal bag as recited in claim 6, wherein the first and second sets of indicia are aligned with one another when said flap is sealed against the rear surface of said envelope to provide a visual indication to the user that a proper seal has been formed.

8. The biodegradable hygiene product disposal bag as recited in claim 1, wherein said means for covering and sealing the open upper end of the envelope with the flap comprises an adhesive layer formed on an inner surface of said flap.

9. The biodegradable hygiene product disposal bag as recited in claim 8, further comprising a release strip for releasably covering the adhesive layer.

10. A biodegradable hygiene product disposal bag, comprising:
an envelope having a pair of longitudinally opposed, closed side edges, an open upper end, and a closed lower end, the envelope defining an open interior region adapted for receiving a used hygiene product;
a flap extending from the open upper end of the envelope, the envelope and the flap being formed from a biodegradable and fluid resistant material;
means for covering and sealing the open upper end of the envelope with the flap; and
first and second sets of indicia formed on an outer surface of the flap and on a rear surface of the envelope, respectively, so that the first and second sets of indicia are aligned with one another when the flap is sealed against the rear surface of the envelope to provide a visual indication to the user that a proper seal has been formed.

11. The biodegradable hygiene product disposal bag as recited in claim 10, wherein said envelope and said flap are formed from low-density polyethylene.

12. The biodegradable hygiene product disposal bag as recited in claim 10, wherein said envelope is substantially rectangular.

13. The biodegradable hygiene product disposal bag as recited in claim 12, wherein each said closed side edge is substantially sinusoidal in shape.

14. The biodegradable hygiene product disposal bag as recited in claim 12, wherein said flap is substantially trapezoidal.

15. The biodegradable hygiene product disposal bag as recited in claim 10, wherein said means for covering and sealing the open upper end of the envelope with the flap comprises an adhesive layer formed on an inner surface of said flap.

16. The biodegradable hygiene product disposal bag as recited in claim 15, further comprising a release strip for releasably covering the adhesive layer prior to usage thereof.

17. A biodegradable tampon disposal bag, comprising:
a substantially rectangular envelope having a pair of longitudinally opposed, closed side edges, an open upper end, and a closed lower end, the envelope defining an open interior region adapted for receiving a used tampon;
a substantially trapezoidal flap secured to the open upper end of the envelope, the envelope and the flap being formed from a biodegradable and fluid resistant material;
means for covering and sealing the open upper end of the envelope with the flap; and
first and second sets of indicia formed on an outer surface of the flap and on a rear surface of the envelope, respectively, so that the first and second sets of indicia are aligned with one another when the flap is sealed against the rear surface of the envelope to provide a visual indication to the user that a proper seal has been formed.

18. The biodegradable tampon disposal bag as recited in claim 17, wherein said envelope and said flap are formed from low-density polyethylene.

19. The biodegradable tampon disposal bag as recited in claim 17, wherein said means for covering and sealing the open upper end of the envelope with the flap comprises an adhesive layer formed on an inner surface of said flap.

20. The biodegradable tampon disposal bag as recited in claim 19, further comprising a release strip releasably covering the adhesive layer prior to usage thereof.
